(19) United States
(12) Patent Application Publication
(10) Pub. No.: US 2008/0219938 A1
(43) Pub. Date: Sep. 11, 2008

(54) SPF compositions

(76) Inventor: Guerry L. Grune, Virginia Beach, VA (US)

Correspondence Address: GUERRY LEONARD GRUNE, 784 S VILLIER CT., VIRGINIA BEACH, VA, 23452, US

(21) Appl. No.: 12/074,906

(22) Filed: Mar. 7, 2008

Related U.S. Application Data
(60) Provisional application No. 60/905,453, filed on Mar. 7, 2007.

(51) Int. Cl. A61K 8/18; A61Q 17/04

(52) U.S. Cl. 424/59

(57) ABSTRACT

A composition comprising; water purified using ozonation, ionization, or distillation or any combination thereof wherein alcohol may be substituted for, or combined with water; at least one emollient including but not limited to chitosan, and aloe vera gel, individually or in any combination; an oil component with spf boosting agents including but not limited to; silicone oil and essential oils, butter milk, waxes impregnated with inorganic sun-block or sunscreen agent and organic/inorganic micronized particles, wood powder and bentonite clay, keratin, either individually or in any combination; at least one inorganic sun-block or sunscreen agent including any metal oxide, glass microsphere, silica and silica compound, and optionally metal oxide pigments with particles that are micronized, submicronized, nanoparticle sized, or otherwise individually or in any combination that can be homogenized in either a water phase, a water-aloe phase, or an oil phase or any phase of the composition; at least one emulsifier such that the emulsifier includes but is not limited to a phospholipid and/or liposome or an aloe vera gel or an ester of coconut oil individually or in any combination, for emulsification of the water, water-aloe, and/or the oil phases. Any of components are preferably mixed using an homogenizer and an appropriate thickening agent including but not limited to xanthan gum, and/or carageenan, either individually or in any combination that is added as required.

### PRIORITY DATA

This application takes priority under 35 USC 119(e) of Provisional Application 60/905,453 filed 7 Mar. 2007 titled “SPF Compositions”.

### FIELD OF DISCLOSURE

This disclosure relates to new and useful ultraviolet radiation protective agents that can be used as beneficial sunscreens and sun-blocks in various compositions or formulations, specifically those of a higher SPF value (15-30, or greater). The compositions include enhanced anti-microbial protection comprising the part contacting the human or animal skin and/or mucosa with a base formulation containing homogeneously dispersed particles of a particle size in the range of 1 to 500 nm in an amount provided on the skin or mucosa.

In addition, the SPF formulations and resulting compositions presented allow for increased immuno-responsiveness as determined by manual muscle testing and more recently with a biophotonic scanner, by providing cytoprotective additives for mammalian skin while also providing avoidance from endocrine disrupting agents—specifically agents that kill cells on contact as determined by LUMI-CELL technology described below. It has been determined that sunscreen agents used in almost all currently marketed and sold ultraviolet protective compositions are essentially void of any cytoprotective agents and essentially all (both active and non-active substances) also contain suspected or documented endocrine disruptive agents—agents that have been proven to kill cells on contact. The term ‘cytoprotective’ refers to the ability to protect cells from becoming pre-cancerous or cancerous.

### BACKGROUND OF THE DISCLOSURE

It has become very evident that excessive exposure of the human skin to sunlight is harmful. It is well documented that human skin, and most likely most mammalian skin, is sensitive to sunlight and artificial light containing radiation of wavelengths between about 290 nanometers (nm) and 400 nm. Ultraviolet radiation of wavelengths between about 290 nm and 320 n (UV-B region) has been known to rapidly produce damaging effects on the skin including reddening or erythema, edema, blistering or other skin eruptions in more severe cases. Prolonged or chronic exposure to radiation in this wavelength range has been associated with serious skin conditions such as actinic keratoses and carcinomas. In recent years, concern has also been expressed regarding ultraviolet radiation of wavelengths above 320 nm (UV-A region) and the adverse effects of such radiation on human skin. The radiation between 320 and 400 nm also contributes to the premature aging of the skin. In addition, recent studies indicate that chronic sun exposure limits the immuno-response of the skin.

A UV protective product could also include known cytoprotective oligosaccharides from aloe barbadensis Miller preventing damage to the skin immune system caused by harmful UV radiation. “Cold-pressed” Aloe which contains the beneficial oligosaccharides and provides an emollient base for the UV protective formulation is possibly the best known choice as a cytoprotective agent that inhibits the loss of skin immuno-competency induced by ultraviolet radiation, as this agent is readily available and comparably inexpensive. Other such inhibitors are not yet well known but it is believed that amino-acids, vitamins or pro-vitamins, nucleo-derivatives, and vegetable extracts, wherein said aminoacids comprise tryptophan, histidine, phenylalanine, tyrosine, said vitamins and provitamins comprise vitamin B6, vitamin A, vitamin E, tocopherols and in particular D-alpha tocopherol, beta carotene, bioflavonoids, nucleotides and polymers thereof, cascara, frangula, camomile, hyperic, calendula, elicriso, licorice or essential oils thereof all may have similar cytoprotective or immune boosting effects on mammalian skin. The essential oils of frankincense and rosemary have been found to work effectively and synergistically in strengthening the neuromuscular response of patients who are exposed to its scent in combination with compositions of the present disclosure. For the purposes of clarifying issues due to the use of essentially any free radical scavenger and/or anti-oxidant compound that is beneficial to human health in combination with the formulations and compositions of the present disclosure, it is known that such compounds are also cytoprotective agents.

One current measure of effectiveness of a sun protective product is indicated by its sun protection factor (SPF). The sun protection factor is the ratio of the amount of exposure (dose) required to produce a minimal erythema reaction in protected skin to the amount required to produce the same reaction in unprotected skin. The absolute dose differs for each human and for each mammal, and is largely dependent on genetic predisposition and ethnic origin of the human. If a human or other mammal would normally require ten minute exposure to sunlight to develop a minimal erythema reaction, then using an SPF 15 sun-block should allow for tolerance of up to 150 minutes of sunlight before developing a minimal erythema. Relatively recent public awareness of the problems of exposure to sunlight has led to a demand for sun-block products with high SPF values, i.e., at or above SPF 8.

What has not been well considered in the sun protection and cosmetics industry heretofore, is the possibility of enhancing the immuno-responsiveness of skin cells to UV light by the proper topical application such as described above by the use of extracts of aloe or similar naturally occurring substances (including kukua nut extract for example or other similar anti-inflammatory agents that are naturally occurring substances). Such substances would preferably not be processed, but if the beneficial anti-inflammatory effects are not lost during processing, then either the processed or non-processed substance may be used. The importance of processing within a short time period after harvesting the aloe plant or other plants/nuts, etc. as well as keeping the plant and subsequent plant extract cool (at or below room temperature) during processing is now well understood. Essential oils including specifically frankincense and rosemary have been shown to have immuno-enhancing properties, as determined by Kinesiologist Dr. John Schmidt of Triangle Wellness Center at 182 Wind Chime Ct. Ste. 203 Raleigh, N.C. 27615. This was determined by a strengthening in neuromuscular response using scent (aroma or aromatherapy) testing of these essential oils. The testing was performed using oils together with compositions of the present disclosure and using the oils either in combination or singularly.

In addition to the determination of immuno-enhancing characteristics using neuromuscular response or “muscle testing”, it has recently become easily possible for humans to accurately measure their skin carotenoid levels, by use of a biophotonic scanner distributed by Pharmanex Corp. The carotenoid levels in the skin are a measure of the immune system's strength, in other words it is possible to achieve and measure the skin's cytoprotective and immuno-enhancing properties by increasing the levels of carotenoids in the skin.

With overexposure to UV light, the human immune system becomes depressed. Most sunscreens further compromise the immune system by including ingredients which lead to the creation of additional free radicals—organic sunscreen compositions that decompose in the presence of UV light. The formulations of the present disclosure, by contrast, contain ingredients which absorb or quench free radicals so that they can not further damage the cells of the skin. The compositions contain ingredients known to boost carotenoid levels, including cold-pressed aloe vera gel and beta glucan, as well as Vitamins C & E, which bind with carotenoids to further boost anti-oxidant levels in the skin.

Carotenoids are a family of natural fat-soluble nutrients important for antioxidant defense (found throughout the plant kingdom. More than 600 carotenoids have been identified in nature but less than 50 are abundant in the human diet. Among these, five carotenoids, b-carotene, a-carotene, lycopene, lutein, and zeaxanthin are found in the blood and known to be important in human health. A large number of epidemiological and experimental studies offer strong evidence that carotenoids are nutritionally important for normal cell regeneration, eye health, plus numerous other health aspects linked to unstable oxygen molecules known as free radicals. Most of the health benefits of carotenoids are associated with their action as antioxidants, that is, they protect cells and tissues from the effects of free radicals. Carotenoids are not soluble in water. Any increase in blood carotenoids is then reflected in an increase of carotenoid concentration in all the organs in the body, which can take up lipoproteins, including skin. Thus, the direct measurement of carotenoids on skin provides information about their levels at “site-of-action”. This is a distinct advantage over measurements which depend only on carotenoids in blood plasma and indicates that our compositions with carotenoids further enhance the body's ability to guard against skin cancer when exposed to high doses of UV light.

Carotenoids can be detected by optical methods, which rely on their different spectral characteristics. However, at the skin surface high concentrations of other pigments such as melanin and hemoglobin interfere in the measurement, making accurate non-invasive carotenoid determinations impossible. Chemical methods like High Pressure Liquid Chromatography (HPLC) and Mass Spectrometry are also important techniques for detecting carotenoids, but unlike optical methods, they are invasive as tissue samples are required.

As an alternative, a new technique called the Pharmanex® BioPhotonic Scanner has been developed based on an optical method known as Resonance Raman Spectroscopy. The scanner measures carotenoid levels in human tissues, eye and at the skin surface using optical signals, called raman signals. These signals identify the unique molecular structure of carotenoids, allowing their measurement without interference by other molecular substances.

There is observational evidence that skin carotenoids as measured by the BioPhotonic Scanner do indeed reflect the body's overall antioxidant defense status and also the relative health of the immune system. The biophotonic scanning methodology allows for current and future development of formulations that are the most comprehensive, scientifically accepted and proven means for providing immuno-enhancing protection from free radical generation due to UV radiation from the sun. Using proper wavelengths emitted by laser or LED or other light sources additional (other than carotenoid) anti-oxidant wavelength specific compound concentrations can also be measured.

This transformation, from qualitative to quantitative measurement of the anti-oxidant levels in any individual's skin, makes it possible to determine the effectiveness of any current or future formulation specifically designed to guard from overexposure to the sun's harmful radiation. Free radicals are scavenged by anti-oxidants which equates to lower exposure to cytotoxic compounds and the ability to maintain healthy skin cells and tissue without suppression of the immune function associated with the same skin cells and tissue. This technique, including measurement and design of new and better sunscreen and sunblock formulations is also a part of the present disclosure.

### Suitable Testing for Endocrine Disruption

In May of 2002, Xenobiotics Laboratories (XDS) of Durham, N.C. submitted preliminary data to ICCVAM for review as a validated regulatory method using their LUMI-CELL™ ER bioassay in response to the Federal Register Notice (Vol. 66, No. 57/Friday, Mar. 23, 2001) as a HTPS method for estrogen active compounds¹⁰. In March of 2004 SACATM gave the LUMI-CELL™ ER bioassay a high priority for validation. In April 2004 the final report on the assay was given to ICCVAM. In March 2005, ICCVAM entered the LUMI-CELL™ ER bioassay into a double blind international validation study using one lab in the European Union, Japan, and the United States. Next, studies were undertaken in which XDS's LUMI-CELL™ ER estrogenic cell bioassay system was used for high throughput screening (HTPS) analysis sunscreens. The results demonstrate the utility of XDS's BG1Luc4E₂ LUMI-CELL™ ER bioassay HTPS system for screening cosmetics for estrogenic/antiestrogenic activity.

There has been a growing need for a fast, reliable, inexpensive method to detect EDCs (endocrine disrupters) in the environment. As part of the present invention, we report a fast, reliable, relatively inexpensive high throughput cell based recombinant bioassay screening method (LUMI-Cell™ ER bioassay) to determine the level of xenoestrogenic EDCs for any cosmetic crème, lotion, paste, etc.

Sunscreen components were purchased from the Inolex Chemical Co., Goldschmidt Chemical Corp., Kobo Products Inc., and Dow Corning. Sunscreens were purchased at Wal-Mart. LUMI-CELL™ ER Bioassay. The BG1Luc4E2 cell line was constructed as previously described by Rodgers and Denison (2000). Briefly, BG1 cells were stably transfected with an estrogen-responsive luciferase reporter gene plasmid (pGudLuc7ere) and selected for using G418 resistance⁹.

Cell Culture and Bioassay Plates. BG1Luc4E2 cells were grown in RPMI 1640. The cells were transferred into flasks containing DMEM media (supplemented with 5% carbon stripped fetal calf serum and G418 sulfate solution), and incubated for four days before harvesting for BG0Luc4E₂ bioassay plates. The cells were then plated in 96 well plates and incubated at 37° C. for 24-48 hours prior to dosing.

Endocrine Extraction Procedure: One gram of each of the lotion components and 0.5 g of each of the sunscreens was placed in MeOH rinsed scintillation vials. Two and 4-gram aliquots of the 3^{rd} Rock Sunblock were also tested. Twenty ml of MeOH was added to each scintillation vial and sonicated for 20 min. Fractions of these extractions, ranging from 1:10 to 1:80,000 were tested. Recoveries were determined using 10 ng 17β-estradiol spiked into 3^{rd} Rock Sunblock prior to extraction with 20 ml MeOH compared to 10 ng 17β-estradiol spiked into 20 ml MeOH. Bioassay Dosing Process. Once the assay plate completed its incubation, the media solution in each well was removed and two hundred microliters of DMEM containing the indicated concentration of the desired chemical to be tested was added to each well. The plate was then incubated for 20 hours before analysis of luciferase activity.

Bioassay Analysis by Berthold Luminometer. After lysing the cells (Promega lysis buffer), the luciferase activity was measured in a Berthold Orion Microplate Luminometer, with automatic injection of 50 microliters of luciferase enzyme reagent (Promega) to each well. The relative light units (RLUs) measured were compared to that induced by the 17beta-estradiol standard after subtraction of the background activity. Each compound was tested at least three times on three different sets of plates and the EC50 value in mmol/ml was determined using the Microsoft Excel Forecast function.

To ensure that our claims have scientific basis and merit, 13 sunscreen products and 8 “non-active” lotion components were tested for estrogenic potency. The samples were tested at 4 g, 2 g, 1 g, 0.5 g, and 0.1 g. The 0.5 g aliquot was selected for sunscreens and 1 g for “non-active” components due to it showing the most activity with the least toxicity. The 3^{rd} Rock Sunblock SPF 30™⁽¹¹⁾ was used as a negative control due to it previously testing as a non-detect. The 3^{rd} Rock Sunblock SPF 30 was also used in recovery determinations. This was performed by dividing the average RLU for the 10 ng 17β-estradiol spiked 3^{rd} Rock Sunblock SPF 30 by the 10 ng 17β-estradiol spiked into 20 ml MeOH. The average recovery was found to be 77.4%. All of the sunscreens detected positive for estrogenic activity with the exception of 3^{rd} Rock Sunblock, which was shown as a non-detect at less than 0.308 pg/g 17β-estradiol equivalent. The sunscreen with the highest estrogenic potential was Coppertone Water Babies SPF45 at 948.66±176.62 ng/g 17β-estradiol equivalent. Based on our test results, the order of estrogenic potency appears to be: Coppertone Water Babies 45>Banana Boat Kids 30>Banana Boat Baby Magic 50>Banana Boat 15>Coppertone SPF 8>Coppertone SPF 30 (Endless Summer)>Hawaiian Tropic Baby Faces 50+>Hawaiian Tropic SPF 8>Coppertone SPF 15>3^{rd} Rock Sunblock SPF 30.

It has therefore been demonstrated that the “non-active” components contribute to a portion of the estrogenic potency of many sunscreen formulations. However, a significant portion of the estrogenic potency remains attributed to the “active” components of the same formulations. Further investigations that include testing “active” and “non-active” components for more detailed analysis regarding estrogenic potency ratios are anticipated. It is apparent from the foregoing results that the test methodology enables one to determine the estrogenic potency of any skin product, not just that of one designed for sun protection. It is known that lotions, shampoos, cleansing agents, cremes, sprays, etc. for human and animal skin contact for various uses, contain numerous endocrine disrupting components. Therefore, this disclosure provides embodiments that include a test methodology to determine levels of toxicity (as defined by killing cells) by determining estrogenic potency and therefore also the propensity for and concentration of endocrine disruption for any lotion, crème, paste, spray, etc. for cosmetic use with or without suncare protection.

The use of silicone oils or other SPF boosting agents, such as the cocoate esters, are useful in providing SPF values of 30 or higher. The well known and commercially available “SPF boosters” have almost without exception proven to be toxic or endocrine disrupters or both and the present embodiments include a scientifically accepted and peer reviewed method to assure the use of only SPF boosters that are neither toxic nor endocrine disrupters.

### SUMMARY OF THE DISCLOSURE

This disclosure relates to new and useful ultraviolet radiation protective agents in combination with lotions, cremes, pastes, sprays, lip balms, cosmetics, etc. that can be used as beneficial sun-blocks and in a specific instance as sunscreens in various compositions or formulations. The compositions include enhanced protection and increased immuno-responsiveness by providing cytoprotective additives for mammalian skin.

It is desirable and provided in the present disclosure to provide sunscreen compositions containing sunscreen agents that overcome the disadvantages of heretofore available compositions and to provide non-endocrine disruptive, adequate, safe protection for mammalian skin while also enhancing the skin's immuno-responsiveness from cancerous or pre-cancerous skin cells in the presence of radiation such as UV light or sunlight.

Another desirable feature of this disclosure addresses the potential risks and disadvantages of currently available commercial products and provides a viable and economically attractive alternative to the present commercial market, and proposes a new and safer rating system to rank these products for the consumer.

Another desired feature of this disclosure is to provide a method and the know-how relating to developing an “all-natural” and primarily all earth-grown ingredient based dispersion of inorganic sun-block agents that will ensure an SPF value of at least 15 or greater. The sunblock may also be translucent or transparent upon application to human skin but at higher SPF levels where higher concentrations of pigments may be necessary, translucency may not be completely achievable. The use of metal oxides and other pigments that assist with matching of skin tone and color is also part of the present disclosure.

The use of a biophotonic scanner allows for measurement of anti-oxidants (in the form of Carotenoids or other beneficial anti-oxidant) concentration levels in the skin to design new and better sunblock and sunscreen compositions is also part of the present disclosure.

The foregoing objects and other features and advantages of the present disclosure are achieved by sunscreen and sun-block compositions containing inorganic sun-block agents or known non-toxic sunscreen agents as the active ingredients. More particularly, the present disclosure relates to sun-block compositions containing zinc oxide and, optionally, titanium dioxide of preferred particle size ranges, and in preferred amounts and ratios. These sun-block agents together with preferably specifically cold-pressed aloe that contains an oligosaccharide of molecular weight of approximately 1-5,000 daltons that is glucose rich and also contains mannose which inhibits the loss of skin immuno-competency helps to form the basis of a novel protective UV formulation. It has become evident that cold-processed aloe that is processed within 45 minutes of harvesting contains about 200 biologically active agents. The synergistic effect of all of these agents is desirable and preferred to further enhance the cytoprotective ability inherent in aloe plant extract. These specific compositions permit the possible use of much lower amounts of the sunscreen active ingredients than previously achievable while still achieving desired and very high SPF values for the compositions and without the unsightly whiteness which occurs in prior sunscreen compositions at concentrations specifically TiO₂ and/or ZnO (even micronized TiO₂) above about 5%. In the sunscreen compositions of this disclosure, considerably higher concentrations of zinc oxide and possibly titanium dioxide may also be used without incurring a whitening effect, e.g., even up to 25% each, with acceptable appearance—meaning little or no whitening effect. Ratios of TiO₂ to ZnO of 1:4 or 1:3.5 using micronized particles have been found to leave little or no unsightly white residue.

Furthermore, our disclosure does not rely upon the use of hydrophilic titanium dioxide preparations as required in other known formulations, nor are energy intensive processes such as powder milling, nor are organic active sunscreens required for high efficacy. Homogenization, however is desirable and necessary for some or all of the necessary mixing steps.

The compositions of this disclosure include compounds, formulas or emulsions that are pastes, lotions, sprays, lip balm, crèmes, and other cosmetic or make-up products, etc. containing at least the following components:
(a) an inorganic sun-block agent including any metal oxide, glass micropheres, silica and silica compounds, and optionally metal oxide pigments with particles that are micronized, nanosized, or otherwise,
(b) an emulsifier or mixtures thereof;
(c) an oil component comprising a carrier oil, preferably an essential oil;
(d) at least one emollient, where the emollient may be the emulsifier of (b) above
(e) sun protective factor (SPF) boosting additives sufficient for imparting an SPF value of 15 or greater
(f) texturing agents providing a powdery feel
(g) optionally carotenoids or other effective antioxidants which are translucent or transparent with respect to the present compositions
(h) if necessary, inorganic pigments for skin color and/or texture matching

The emollient or emulsifier can be aloe as it is “cold pressed” or an extract of aloe that is currently removed during normal processing and recovered by some means. The aloe or its extract may not provide sufficient emulsification with regard to the remaining ingredients of the composition. It has since been determined that a single species of aloe is preferred and best for providing a healthy and well dispersed product with the highest known concentrations of cytoprotective agents—aloe barbadensis Miller-Stockton. Aloe is available in gel and in oil forms. It may also be possible to use a liposome or phospholipid such as Phosphatidyl-choline (PC), as described below.

An emulsifier (also known as a surfactant from surface active material or emulgent) is a substance which stabilizes an emulsion. An emulsion is a mixture of two immiscible (unblendable) substances. One substance (the dispersed phase) is dispersed in the other (the continuous phase). Examples of emulsions include butter and margarine, mayonnaise, the photo-sensitive side of film stock, and cutting fluid for metalworking. In butter and margarine, a continuous lipid phase surrounds droplets of water (water-in-oil emulsion). Emulsification is the process by which emulsions are prepared. Examples of emulsifiers are egg yolk (where the main emulsifying chemical is the liposome or phospholipid lecithin), and mustard, where a variety of chemicals in the mucilage surrounding the seed hull act as emulsifiers; proteins and low-molecular weight emulsifiers are common as well. Whether an emulsion turns into a water-in-oil emulsion or an oil-in-water emulsion depends of the volume fraction of both phases and on the type of emulsifier. Generally, the Bancroft rule applies: emulsifiers and emulsifying particles tend to promote dispersion of the phase in which they do not dissolve very well; for example, proteins dissolve better in water than in oil and so tend to form oil-in-water emulsions (that is they promote the dispersion of oil droplets throughout a continuous phase of water).

Emollients are substances which soften and soothe the skin. They are used to correct dryness and scaling of the skin. The terms ‘moisturizer’ (to add moisture) and ‘emollient’ (to soften) are sometimes used interchangeably as they describe different effects of these agents on the skin. However, the term emollient is most often used to describe single ingredients, whereas ‘moisturizer’ describes finished products. Emollients have three basic actions: 1) Occlusion—providing a layer of oil on the surface of the skin to slow water loss and thus increase the moisture content of the stratum corneum. 2) Humectant—increasing the water-holding capacity of the stratum corneum. 3) Lubrication—adding slip or glide across the skin.

An example of an emollient that will boost the occlusivity of the present disclosure is chitosan. Chitosan is a linear polysaccharide composed of randomly distributed β-(1-4)-linked D-glucosamine (deacetylated unit) and N-acetyl-D-glucosamine (acetylated unit). Chitosan is produced commercially by deacetylation of chitin (can be produced from chitin also), which is the structural element in the exoskeleton of crustaceans (crabs, shrimp, etc.). Chitosan enhances the transport of polar drugs across epithelial surfaces, and is biocompatible and biodegradable. Purified qualities of chitosans are available for biomedical applications. Chitosan and its derivatives such as trimethylchitosan (where the amino group has been trimethylated) have been used in non-viral gene delivery. Trimethylchitosan, or quaternised chitosan, has been shown to transfect breast cancer cells. As the degree of trimethylation increases the cytotoxicity of the derivative increases. At approximately 50% trimethylation the derivative is the most efficient at gene delivery. Oligomeric derivatives are relatively non-toxic and have good gene delivery properties. Aloe acts as an emollient in the present compositions as well.

The compositions of this disclosure provide formulations having an SPF of at least 10, with titanium dioxide, zinc oxide, or a combination of the two (with or without silica or silicon dioxide and/or cosmetic glass microspheres, or other metal oxides including but not limited to magnesium, manganese, iron, copper, etc.), with a treated or untreated hydrophilic surface, at concentration levels of at least 4% and preferably at least 20% to reach SPF 15 or greater. The compositions of this disclosure exhibit extremely efficient uses of sunblocking components, particularly zinc oxide. Alternatively, higher levels of preferably micronized titanium dioxide or zinc oxide can be used with or without ultramarine pigments added to the composition. These pigments are known to eliminate the whiteness and poor spreadability of currently available compositions. The sun-block agent inorganic/organic dispersion can be made in the following way:

Essentially, the compositions of this disclosure are easily made by homogenization (high speed/shear mixing) and provide an excellent dispersion of the inorganic based sunscreen/sunblock agent throughout the composition, thus ensuring even skin coverage. With or without the use of ultramarine pigments, the compositions are substantially invisible upon application to the skin.

Thus, in one possible embodiment, the present disclosure is directed toward a colored sunscreen emulsion comprising:
(a) at least one ultramarine pigment that imparts a color other than white to the emulsion with a titanium dioxide or zinc oxide or possibly fumed or fused silica or even silicon dioxide or micronized glass cosmetic spheres so that when the emulsion is rubbed into the skin, the color substantially disappears;
(b) at least one sunscreen active agent in an amount effective to protect skin against the actinic radiation of the sun—this preferably being ZnO or Z-Cote® (micronized particles—preferably nanoparticle sized to assure transparency);
(c) a cytoprotective substance such as a glucose-rich mannose-containing oligosaccharide obtained from and used with aloe barbadensis Miller as the at least one emulsifier; and
(d) sufficient water to form the other than a white colored emulsion; and sufficient dispersion to assure SPF of at least 15 and an SPF booster that shows no appreciable toxicity.

The amount of the ultramarine pigment in the composition can range from 0 to about 25 weight percent of the composition, and preferably from about 1 to about 5 weight percent of the final formulation.

An additional embodiment includes a composition comprising;
(a) water purified using ozonation, ionization, or distillation or any combination thereof wherein alcohol may be substituted for, or combined with water;
(b) at least one emollient including but not limited to chitosan, and aloe vera gel, individually or in any combination;
(c) an oil component with spf boosting agents including but not limited to; silicone oil and essential oils, butter milk, waxes impregnated with inorganic sun-block or sunscreen agent and organic/inorganic micronized particles, wood powder and bentonite clay, keratin, either individually or in any combination;
(d) at least one inorganic sun-block or sunscreen agent including any metal oxide, glass microsphere, silica and silica compound, and optionally metal oxide pigments with particles that are micronized, submicronized, nanoparticle sized, or otherwise individually or in any combination that can be homogenized in either a water phase, a water-aloe phase, or an oil phase or any phase of said composition;
(e) at least one emulsifier wherein said emulsifier includes but is not limited to a phospholipid and/or liposome or an aloe vera gel or an ester of coconut oil individually or in any combination, for emulsifying said water, water-aloe, or oil phase;
and wherein any of components (a)-(e) are preferably mixed with an homogenizer and wherein an appropriate thickening agent including but not limited to xanthan gum, carageenan, either individually or in any combination is added as required.

Optionally, the present compositions can contain one or more additional ingredients, including emollients, waterproofing agents, dry-feel/powdery-feel modifiers, insect repellants, antimicrobial preservatives and/or fragrances.

In another embodiment, the present disclosure is directed towards a method for protecting the skin against sunburn while increasing mammalian skin cell immuno-response to cancerous skin cells while eliminating possible endocrine disruption response of human organs comprising topically applying the sunscreen formulation, as described above, to the skin.

An advantage of the present disclosure is that it provides a sunscreen and a method for protecting against sunburn that enables the user to apply the sunscreen more completely and uniformly to the skin, thus providing more effective protection against skin damage and homogenously enhancing cytoprotection while eliminating endocrine disruptive organics, thus providing for long term health and safety in the presence of UV light.

Another advantage of the present disclosure is that it provides a sunscreen with a color indicator which has a low fabric staining potential, and for which those stains that form can easily be removed from fabrics.

Still yet another advantage of the present disclosure is that it provides an optionally colored sunscreen and a method for protecting against sunburn that is more enjoyable for human use because of the attractiveness and appealing nature of the color indicator.

For domesticated animals, the use of matching colors may also be appealing.

This disclosure allows for the use of ultrafine ZnO particles that are invisible when applied to human skin—this is normally less than 0.5 microns and most often at less than 0.2 microns. This “invisible” ZnO would be the primary and perhaps only sunblock “active” ingredient or could be combined with titanium dioxide and silica or silicon dioxide and cosmetic microspheres and optionally inorganic pigments to enhance dispersion when mixed with homogenization equipment and therefore provide a higher SPF value.

### DETAILED DESCRIPTION OF THE DISCLOSURE

The UV-protective compositions of this disclosure yield highly effective ultraviolet (UV) blocking capabilities. A typical titanium dioxide sunscreen composition of SPF 15 requires levels of titanium dioxide that impart a significant whitening effect to the skin; the compositions of this disclosure minimize this disadvantage and are therefore also economically viable to produce. Use of micronized titanium dioxide which is submicron sized has also been used in formulating the compositions of the present disclosure.

The composition of this disclosure include emulsions that are cosmetically superior to conventional inorganic preparations, including water-in-oil titanium dioxide-only formulations, at equivalent SPF ratings, due to the method and type of dispersion as described above. The compositions of this disclosure can be used for sun protection in daily wear or facial products as well as for recreational situations. Because of the efficiency of the system, the inventive formulations are significantly better than the prior art in that they do not provide for additional toxic or cell killing agents to be included in either the active or inactive list of ingredients. There are several ingredients that contribute to the unexpectedly high efficiency of the compositions blocking of UV radiation. It has been found, however, that only one known organic UVA protector, butyl-methoxydibenzoylmethane has been shown to be benign regarding activity in cells or developmental effects on animals. Depending on the need for individual formulations based on the inventive concept herewithin, the use of this or other UVA protectors may be required. The formulations of this disclosure is intended to filter harmful UVA as well as harmful UVB radiation so that the skin is fully protected, but primarily are designed to block light. As each mammal's immuno-response system and skin composition is different, the required amounts required for application to the skin will vary. In addition, the actual UV protective formulation will vary based on the environmental location, length of exposure, age, health and other factors involving individual mammals, such that the concentrations of UVA screens, UVB screens, inorganic pigments, and cytoprotective agents will vary.

### Natural Muscle Testing

Another feature of this disclosure is that the compositions not only protects the wearer from the harmful effects of the sun but actually strengthens the wearer's ‘Neuro-muscular response’. One test method, ‘Applied Kinesiology’, has been used to test a user's neuro-muscular response to sunblock. Applied kinesiology (AK) is a form of diagnosis using muscle testing as a primary feedback mechanism to examine how a person's body is functioning. The compositions of the present disclosure have been tested according to this response and all patients have been “strengthened” in response to the inventive composition being applied to their skin. This science is still evolving and is not fully understood and the evidence is empirical, but this testing has been performed over the course of 2 years (2004-2005) at Dr. John Schmitt's office (Triangle Wellness Center at 182 Wind Chime Ct. Ste. 203 Raleigh, N.C. 27615) and is evidentiary of the fact that the composition is, in fact, “immuno-enhancing”.

Manual muscle testing (MMT) was born in the 1950s with the work of two physical therapists (Kendall and Kendall) and their historic text, Muscles: Testing and Function. Since that time, this body of knowledge has been integrated into the standard procedures of orthopedists, neurologists and practitioners of physical medicine worldwide.

In 1964, Goodheart began utilizing the methods of Kendall and Kendall in clinical practice to assess patterns of functional muscular inhibition (“weakness”) and determine which treatment options, based on known neurophysiologic parameters, might be viable to restore proper muscular facilitation (‘strength’). His clinical research marked the beginning of applied kinesiology (AK).

Just as a DTR represents a “snapshot” of a spinal cord reflex loop, a manual muscle test is also a “snapshot.” It is a clinical window of the central nervous system which, due to its plasticity, is constantly in flux. The only difference is that the manual muscle test is exceedingly more complex, not only in terms of its mechanism, but also in how well a doctor can be trained to perform it. That is where the science and the art must blend. The experienced neurologist will likely be more successful at eliciting an accurate DTR, and appropriately interpreting it in the context of an exam, than the family practice physician. By the same token, expert applied kinesiologists are better equipped with the psychomotor skills necessary to perform accurate MMT. Manual muscle testing as a diagnostic tool is a relatively new innovation. In addition to the previous uses, manual muscle testing has been fine-tuned to test almost every muscle in the body for function. When testing a specific muscle (as in Applied Kinesiology), or using a general muscle group (as in Total Body Modification or NeuroEmotional Technique), the doctor evaluates the functional neurology of that muscle or muscle group. The function of the muscle and the result of the muscle test will assist the doctor in determining the proper treatment protocol. The technique's effectiveness seems to be related to using energy reflex points, somewhat similar to acupressure, at specific body sites to neutralize the energy imbalance in the body. There also seems to be a profound normalization that rebalances the autonomic nervous system. The autonomic nervous system is frequently referred to as the master regulator of metabolism because in controls all the involuntary activities of the body, all the functions of the body that are not under your conscious control.

This would include functions such as heart rate, digesting, breathing, tissue repair and rebuilding, regulation of your body temperature, immune system and countless other functions. Once the autonomic nervous system is rebalanced and improved, many physical aspects of disease have a corresponding improvement.

### Use of a Biophotonic Scanner

The use of a biophotonic scanner allows for measurement of anti-oxidants (in the form of Carotenoids or other beneficial anti-oxidant) concentration levels in the skin to design new and better sunblock and sunscreen compositions is also part of the present disclosure as has been detailed already in the Summary section above.

A more complete rating mechanism than the SPF rating method is suggested here. The immuno-response rating system could be a simple 0-10 value, with 10 indicating a substance within a UV-protective composition that is most beneficial to boosting skin cell immune responsiveness to carcinoma, melanoma, etc. (for instance—specifically cytoprotectiveness).

Therefore, as part of the present disclosure, a rating system for UV-protective compositions is proposed that includes;
SPF value—15 or greater desired
Immuno-responsiveness factor or cytoprotectiveness (IRF)—5 or higher desired (greater than 0)

This rating system has particular relevance to the newly discovered methods reported here required to;
1) measure in a qualitative or quantitative manner and
2) process a dispersion capable of ensuring an SPF 15 or greater value without sacrificing the need to retain an “all earth grown” or “all natural” composition and keeping the composition transparent or matching in color and tone with the skin to which it is applied. The texture should also be appealing in that the composition provide a “powdery, light” feel and absorbs quickly and easily into the pores of the skin.

The ultimate UV-protective formulation would safely block and/or screen UV light, enhance the immune responsiveness of the skin in the absence or presence of UV, and ensure the user that there is no toxic substance present. This is the goal of the compositions presented in this disclosure.

Ease of application and cosmetic appeal, on the other hand, are also important in formulating sunscreen compositions. These characteristics rely on subjective evaluations such as visual and tactile impressions by the user. Consumer research studies indicate that a sunscreen formulation should rub in easily, leave the skin non-sticky and, above all should be invisible or at least translucent to match skin color of the skin after application. Sunscreen compositions containing organic sunscreen agents have been found, in some cases, to irritate the skin. A recent development in the reduction of particle sizes of ZnO has resulted in microfine essentially clear ZnO when applied to the skin. Formulation in the family known as Z-Cote® which is a trademarked composition sold by BASF is one such example of a micronized zinc oxide available today. (The process of micronization refers to breaking up a substance into particles that are only a few micrometers in substance—or submicrometer in size.) The groups of inorganic sun-block agents includes titanium dioxide, micronized titanium dioxide, zinc oxide, micronized zinc oxide, iron oxide, silicon dioxide, magnesium oxide, manganese oxide, silica, alumina, and aluminum oxides and other metal compounds that can provide safe and effective sunblocking compositions such as those using Boron. Cosmetic microspheres, such as those made of silica dioxide or silica such as CM-111 AS produced by 3M Corp of St. Paul, Minn., can also be used as an inorganic sun-block agent.

In addition, the need for an acceptable emollient that reduces the negative affects associated with abrasive inorganics and that also includes the benefit of providing cytoprotection and healing of the skin is necessary. Allowing for the reduction of irritation or sensitization of the skin suggests that “cold-pressed” Aloe and Chitosan are useful ingredients for such a UV-protective formulation.

It has also been determined that it is quite difficult, if not impossible, for current dispersion systems of micronized TiO₂, ZnO, SiO₂ and the like to be non-toxic. As presented in earlier disclosures, the Lumi-Cell test technique (developed by Xenobiotics Laboratories of Durham, N.C.) can be used to determine if human cells are killed. Therefore, in essence, using one of several definitions of toxicity—adverse effects occurring as a result of repeated daily dosing of a chemical or exposure to the chemical, for part of an organism's lifespan—the dispersions themselves are toxic. The present disclosure includes the possible use of aloe, not only as an emollient, but also as a very effective dispersing agent for the inorganic micronized (and larger) sunblock active agents. High speed shearing (accomplished with or without the use of an homogenizer), followed by high speed mixing (up to 40,000 rpm with an homogenizer) provides a consistent, usable, and easily blendable inorganic/organic dispersion free of any known toxic substances (if the aloe source and inorganic particle source is well documented and controlled). A well mixed and homogeneous dispersion is essential in providing sufficient homogeneity and SPF values with any associated cream, lotion, gel, spray, etc. that is used to provide a formulation consistent with the basis of the present disclosure. It has been found recently that the use of an homogenizer is essential to prevent separation of (primarily oil-based) components during extended shelf-life and that there are synergistic effects both in terms of transparency and SPF boosting capabilities in the final compositions that also require homogenization.

### Non-Toxic SPF Boosting Agents

To provide the proper SPF value, it is also necessary to enhance or boost the SPF number using boosting agents. These boosting agents should also not be toxic (cell-killing). It is likely that many natural oils and perhaps derivatives of other natural occurring substances (such as essential oils of safflower, sunflower, rice bran, eucalyptus, rosemary, peru balsam, olibanum, orange, almond, sesame, ylang ylang, jojoba, or coconut) that can provide dispersion capabilities to enhance or boost SPF values may also be determined to be toxic free and are therefore also part of this disclosure. It has been determined that to increase SPF values for both in vivo and in vitro testing, film forming properties are important. The following film forming agents may also be used in the formulations and resulting compositions of the present disclosure: wheat protein extract, silk protein, galactoarabian (aribinogalactan), marine collagen, pea extract, purcellin oil, preen oil, and wild mango butter.

Bentonite can be used to boost SPF values. Colloidal Bentonite contains the active constituent montmorillonite super-refined with demineralized water as a vehicle. The liquid bentonite was the first of its kind to be processed removing the dirt, mica and impurities leaving the active ingredient Montmorillonite in a colloidal suspension.

Aloe Vera gel serves numerous purposes in the present disclosure, including acting as a dispersant, as an emollient, boosting the SPF value, and improving aesthetics, and is believed by many to have healthful benefits.

A preferred embodiment of the present disclosure includes the use of a pure strain of aloe-vera known as aloe barbadensis Miller-Stockton. This strain or species of aloe is believed to have low concentrations of the enzyme aloin. Aloin is an enzyme which when taken internally has a diuretic effect (i.e. it causes diarrhea) by causing inflammation within the human intestinal tract. The Stockton strain is believed to be low in aloin because the product is marketed for internal consumption and has not had any documented diuretic effect on thousands of users over the course of more than 30 years. Further, the Stockton strain is believed to include a greater concentration of cytoprotective oligosaccharides. Utilizing the Stockton strain of aloe for the formulations of the present disclosure ensures purity, uniformity, and a proper medium for dispersing the active inorganic sun-block agents. Further, the Stockton strain is ideal because it is a single species source and therefore reproducible on a batch-to-batch basis. The Stockton strain is not mixed with any other strains of aloe which are known to possess large doses of aloin or other impurities including toxic and even poisonous constituents if consumed. Further, the Stockton strain is ‘cold-pressed’ mechanically and not processed chemically by carbon adsorption or any other chemical means. The aloe processing industry includes carbon adsorption to prevent color loss. However, the carbon adsorption process also removes some or all of the cytoprotective oligosaccharides which the present disclosure requires. By using a single species of aloe, it is also possible to maximize the most advantageous health features of the plant (minimize any unhealthy features) used in any of the compositions of the present disclosure.

### Compositions

The compositions of this disclosure may, include one or more of a select group of anionic emulsifiers. In particular, salts of certain fatty acids are useful in the formulations of this disclosure, preferably salts of saturated fatty acids and/or salts of straight-chain fatty acids. Alkali metal salts and alkali earth metal salts and salts with amine groups are more preferable for use in the compositions of this disclosure. For example, stearic acid and its salts are useful as emulsifiers in the compositions of this disclosure, while the use of isostearate salts tends to produce a composition which is not very efficient in the use of sunscreen. Likewise, oleate salts are not useful as they are unsaturated and do not result in efficient sunscreen compositions. Sodium borate is an example of a preferred salt.

The emulsifier may also be sodium stearate. While it is not fully understood why some salts of fatty acids result in an improved inventive composition, it is theorized that salts of straight-chain fatty acids, (the fatty acids having a relatively high melting point, above 70° C. or higher), are preferable due to their structure.

The anionic emulsifiers should be present in the compositions of this disclosure in an amount from about 0.01 to about 10%, more preferably 0.1 to about 7% and most preferably from about 0.5 to about 5%. There are additional emulsifiers present in the compositions of this disclosure. However at least one anionic emulsifier should be present in order to achieve the products of this disclosure. The fatty acid salt emulsifiers may be added to the composition as the salts, or the salt may be formed in situ.

Phosphatidyl-choline (PC), a phospholipid also known as lecithin and PhosChol, can be used as a natural dispersant and/or emulsifier. Some commercially available PC products are Phospholipon 90G® and Phospholipon 85G®, distributed by the American Lecithin Company of Oxford Conn. PC can be dispersed into an oil, glycerin, aloe vera, or otherwise suitable solvent before being added into the present formulations of the present disclosure as an emulsifier or dispersant. A liposome is a spherical vesicle with a membrane composed of a phospholipid bilayer used to deliver drugs or genetic material into a cell. Liposomes can be composed of naturally-derived phospholipids with mixed lipid chains (like egg phosphatidyl-ethanolamine), or of pure components like DOPE (dioleolylphosphatidylethanolamine). The lipid bilayer can fuse with other bilayers (e.g., the cell membrane), thus delivering the liposome contents. By making liposomes in a solution of DNA or drugs, (which would normally be unable to diffuse through the membrane), they can be (indiscriminately) delivered past the lipid bilayer. Liposomes can be used as emulsifiers in the same manner as the phospholipids discussed above.

Humectants can form an important part of the compositions in the present disclosure. The main purpose of any cream is to keep the skin moist. Collagen, elastin, panthenol (pro-vitamin B5) and keratin enjoy some popularity as humectants. Another example is Pepha®-Nutrix, a product of Pentapharm Ltd of Basel, Switzerland. Natural phospholipids, or lecithin, are also an excellent humectant. An important benefit of phospholipids is that they are hygroscopic (attract water from the surrounding air) and hold water where an increased level of hydration is needed. Therefore, phospholipids increase the hydration levels of the skin without being occlusive (forming a film to prevent water loss, and preventing normal cellular function).

To enhance the “powdery feel” earlier presented, it is possible to include Tapioca as well as sodium borate and/or glass microspheres and/or powdered milk or butter milk and/or sucrose stearate or sodium stearate and/or wood powders and clays (such as bentonite). The amount needed to ensure the powdery texture together with the proper viscosity will vary from formulation to formulation depending, among other items, on mixing sequence, homogenization of various oil/water/aloe components, temperatures, etc.

A carrier oil is useful in the compositions of this disclosure. There are a range of different carrier oils each with their own individual properties and suitability for aromatherapy. The carrier oil may be selected from the group of essential oils or other known non-toxic esters. Other carriers include castor oil, avocado oil, broccoli seed oil, and even keratin, and micronized or colloidal bentonite can be used as a carrier.

The carrier oil is more preferably an essential oil, and should be present in the composition in an amount of between about 0.1% and about 10%. More preferably, it should be present in the amount of between about 1% and about 5%. Most preferably, it should be present in the amount of between about 2% and about 4%. Examples of essential oils include oils of jojoba, rice bran, sesame, safflower, almond, sweet almond, eucalyptus, sunflower, peru balsam, rosemary, olibanum, orange, sunflower, ylang ylang, apricot kernel, avocado, borage, cocoa butter, evening primrose, grapeseed, hazelnut, kukui, macademia nut, olive, peanut, pecan, rose hip, bergamot, jasmine, neroli, patchouli, petitgrain, rose, vetiver, chamomile, mandarin, lavender, grapefruit, cypress, bay laurel, frankincense, clary sage, ginger, helichrysum, lemon, sandalwood, basil, black pepper, peppermint, geranium, wintergreen, thyme, tea tree, tangerine, spearmint, common sage, rosewood, pine, patchouli, oregano, nutmeg, myrrh, melaleuca, marjoram, manuka, lemon grass, lavender, juniper, ginger, cumin, clove, camphor, bay leaf, anise, allspice, and hyssop. A number of the above mentioned essential oils, including jojoba and avocado, can be also utilized in the present formulations also as emollients.

For conventional UV-protection formulations, an oil phase should contain at least two materials, the carrier oil or essential oil and a conventional emollient known to those of ordinary skill in the art as useful in sunscreen products, such as mineral oils, ester oils, vegetable oils, emollients such as fatty acid esters and the like. For the present disclosure, the use of a cold pressed aloe barbadensis Miller and specifically the Stockton species is to be substituted as an emollient or can be used in combination with the oils or emollients that are proven to be non-endocrine disrupting as well as not interfering with augmenting the cytoprotective enhancing effects of the known effective oligosaccharide aloe extract. The emollient should be present in the formulation in a ratio to the carrier concentration of from about 1:1 to about 3:1, most preferably, about 2:1. The carrier oil and the emollient should comprise from about 2% to about 40% of the total composition weight.

A third element which should be present in the compositions of this disclosure is an inorganic sunscreen compound, such as titanium dioxide, zinc oxide or combinations thereof. Possible other inorganics include the use of fused or fumed silica or even silicon dioxide. Preferably, titanium dioxide, zinc oxide, silica, silicon dioxide, and/or cosmetic glass-based microspheres should be used having a primary particle size of less than about 300 nm in diameter. Larger particle sizes may also be used if necessary along with special pigments to ensure transparency or at least skin color matching. The inorganics should be present in the composition in the amount of from about 2% to about 25%. More preferably, it should be present in the amount of from about 2% to about 20%. The inorganic sunscreen compound should be oil dispersible, and may be present with or without surface coating.

The ratio of titanium dioxide or zinc oxide to the weight of the carrier oil and the emollient combined should be from about 0.0:1 to about 1:1. More preferably, the ratio should be between about 0.25:1 or 2:3, and most preferably 0.33:1, to maintain transparency or translucency upon application to the skin.

In the case where salts of fatty acids are used, care should be taken to keep the pH of the compositions of this disclosure at a level above about 5, more preferably, above about 5.5. Maintaining the pH at this level will ensure that these anionic emulsifiers remain in the salt form, which is important in retaining the stability and efficacy of the composition.

Additionally, the usual elements of a modern sunscreen emulsion system may be necessary such as a polymeric thickener/stabilizer, one or more additional emollient oils, microbial preservatives, waterproofing agents, antioxidants, fragrance, humectant, and of course the water vehicle may all be utilized using careful selection or restraint based on the constraints of providing a non-toxic immuno-enhancing composition.

Base formulations of this disclosure may also be used as carrier compositions for active topical agents having dermatological effects, including depigmentation agents, anti-aging ingredients, anti-fungal agents, anti-microbial agents, and the like. For example, depigmentation agents can include magnesium ascorbyl phosphate or hydroquinone. Anti-aging agents can include retinoid compounds and alpha-hydroxy acids. Anti-fungal agents that can be included in the compositions of this disclosure include azole compounds including ketoconazole and the like. Anti-microbial agents may include triclosan.

The compositions of this disclosure would then have minimally a multi-action capability, as they would contain both sunscreen agents and other actives for protecting, treating, and enhancing the immuno-responsive nature of the skin, providing anti-aging properties, as well as ensuring necessary shelf life by providing anti-bacterial activity.

The compositions of this disclosure can be incorporated into various cosmetic and personal care products such as hand and body lotions, oils, ointments, lip balm products, facial cosmetics and the like.

One of the major challenges in providing the composition of the present disclosure is to provide a non-toxic, immuno-enhancing high (15 or greater) SPF formulation that can be readily achieved in a manufacturing environment for a reasonable cost. The use of aloe as both an emollient and a surfactant/dispersion agent together with either ZnO, titanium dioxide, silicon dioxide, fluoropolymers, silica, magnesium, glass microspheres, clays, wood powders, etc., micronized or otherwise (inorganic or acceptable organic sun-block agents) with optional additions of anti-oxidants such as caretonoids and dispersing agents including phosphotidyl choline or phospholipids, or liposomes or keratins in the manner detailed above and claimed has not been heretofore accomplished. The addition of SPF boosting agents that are not toxic is also unique to this disclosure and has heretofore not been previously considered or explored.

It should be emphasized that SPF values of 15 or greater can be achieved solely by blending and subsequent mixing of aloe with vegetable glycerin (or glycerol as it is also known) and zinc oxide (micronized in this case) and an oil and that we have achieved a superior product using this technique. This would be the so called “aloe-water” phase that would be subsequently mixed at high speed with the so-called “oil-phase”. Blending or homogenizing would be accomplished using either the aloe-water phase or oil phase and in so doing, the aloe would not be necessarily diluted with water until after the full addition and blending of the inorganic sun-block agents. Homogenization may be used to provide SPF values higher than 15. Water dilution during or after blending is acceptable but not necessary and in some cases it may be undesirable. It is also desirable to add additional inorganic sunblocking agents directly to the oil phase to insure SPF values greater than 15. Often, it is necessary to add the inorganic sunblocking agents to both phases (oil and water) to provide a superior formulation.

The aloe and specifically single species of aloe as described above, seems particularly well-suited (with and without the use of glycerin) to provide an emulsion that is homogeneous and can achieve sufficient SPF values using 14% or more (by weight) of the inorganic sun-block agents. Ratios of 4 or 3.5:1 or 3.3:1 (weight percent) of ZnO to titanium dioxide (both in the micronized version) have been used to achieve SPF 30 or more. Micronized sun-block agents are best for this emulsion as they provide the best surface area-volume ratio for proper wetting of the ZnO and other micronized inorganic/organic particles.

The well known and commercially available “SPF boosters” have almost without exception proven to be toxic such that the present disclosure provides for the use of only SPF boosters that are not toxic. The use of phospholipids or liposomes described above may also provide the required oil-water dispersion and thus also boost SPF.

Below are listed additional components which have been or could be added to the basic SPF composition to boost immuno-responsiveness, SPF values, and cytoprotectiveness without introducing toxicity (human cell killing agents).

It is known that the use of green tea extract may be effective in reducing sunburn. Green tea inhibits UVB-induced erythema response in the skin (redness reaction). At the same time it supports the production of melanin, the skin's own natural sunburn protection. Thus green tea helps reduce the risk of sunburn and boosts SPF.

Tocopherol, or Vitamin E oil, is a fat-soluble vitamin in eight forms that is an important antioxidant. Vitamin E is often used in skin creams and lotions because it is believed to play a role in encouraging skin healing and reducing scarring after injuries such as burns. Natural vitamin E exists in eight different forms or isomers. Each form has its own biological activity, the measure of potency or functional use in the body. For the present disclosure, the most stable forms of Vitamin E are desired.

Rosehip, also called the rose haw, is the pomaceous fruit of the rose plant and a powerful antioxidant. Rosehip can be used as an emollient in the present disclosure. The use of vitamin C (ascorbic acid or other available forms of Vitamin C) in sunscreen or sunblock formulations should be in a stabilized form such as Magnesium ascorbyl phosphate. For the present disclosure and associated formulations, the most stable form of Vitamin C is optionally incorporated. Keratins may provide an SPF boost to the present compositions. Keratins contain a high proportion of the smallest of the 20 amino acids, glycine, whose “side group” is a single hydrogen atom; also the next smallest, alanine, with a small and uncharged methyl group. Sucrose stearate is usually a white or light brown block or powder, with little or no smell and no taste. Because it is made from vegetable sources it is completely biodegradable. One commercially available form of sucrose stearate is Crodesta® F-160, manufactured by Croda of Yorkshire, England.

Lanolin is a thick natural moisturizer to soothe and protect skin. Lanolin forms an emulsion with water that's easily absorbed by the skin, softening it and preventing it from frying and cracking. It is used for dry skin, sunburn, and windburn, and should also boost SPF.

A number of oils are used in commercial sunblocks as SPF boosters. Such oils may be effective at boosting SPF on their own in some cases, or in combination (synergistically) with other oils in other cases. Among these oils are sunflower oil, safflower oil, almond oil, rice bran oil, eucalyptus oil, sesame oil, orange oil, jojoba oil, rosemary oil, peru balsam oil, grape seed oil, pomegranate seed oil, broccoli seed oil, macadamian nut oil etc. Certain waxes may also have a positive SPF effect, including beeswax, orange wax, synthetic waxes and the like.

Beeswax is a product from a bee hive and is a tough wax formed from a mixture of several compounds; its main components are palmitate, palmitoleate, hydroxypalmitate and oleate esters of long-chain (30-32 carbons) aliphatic alcohols, with the ratio of triacontanylpalmitate CH₃(CH₂)₂₉O—CO—(CH₂)₁₄CH₃ to cerotic acid CH₃(CH₂)₂₄COOH, the two principal components, in a ratio of 6:1. Beeswax is used commercially to make fine candles, cosmetics and pharmaceuticals including bone wax (cosmetics and pharmaceuticals account for 60% of total consumption), in polishing materials (particularly shoe polish), as a component of modelling waxes, and in a variety of other products. For the present disclosure, the use of Hydroxyoctacosanyl hydroxystearate is not preferred but may have to be used as a beeswax substitute as a consistency regulator and emulsion stabilizer due to recent decreases in the bee population in North America. Beeswax's primary use in the present disclosure is to increase the water-resistant capabilities of the composition. The beeswax can also be impregnated with sun-block materials (micronized zinc oxide and titanium dioxide, etc.) in order to prevent these materials from being easily washed away during use. High altitude beeswax is preferred as the SPF value is higher for this substance from bees living at elevations well above sea level. Other products from bees including bee pollen and propolis as well as flower pollen are useful to impart powdery feel as well as boost SPF values for the present compositions.

The preferred preservative in the present disclosure is Biovert®, a product of Arch Chemicals®. Biovert® is a system of two linked preparations, which by themselves do not offer antimicrobial efficacy, but together offer anti-microbial efficacy. Biovert® mimics a naturally occurring antimicrobial-antioxidant protection system. When the two-part system is combined, a cascade of linked reactions takes place to generate antimicrobial products in situ. The cascade is initiated by the action of the glucose oxidase enzyme in the presence of its substrate (glucose) and oxygen. This generates H₂O₂, which is used by the lactoperoxidase to catalyze the oxidation of I⁻ and SCN⁻ anions, forming hypoiodite and hypothiocyanate which have antimicrobial activity. The result is rapid microbial cell death. Other natural preservatives include tea tree and thyme essential oils, grapefruit seed extract, and D-alpha Tocopherol Acetate (Vitamin E). Both ZnO and titanium dioxide are anti-bacterial agents and will aid in the ability to preserve the compositions of the present disclosure. One additional antimicrobial agent that comprises an extract of aralia cordata of plant with a lower alcohol and ethyl alcohol may also be used. The minimum amount of the active ingredient of the extract of aralia cordata is in a range of 0.005 wt. % to 0.08 wt. % for our compositions.

One possible method which is presented as an example of producing formulations for composing the sunblock composition of the present disclosure, may be performed using a two-vessel method, in which the oil and aloe or water or aloe/water phases are individually prepared. This process produces a smooth, uniform, white to light ivory emulsion, especially in conjunction with the use of an homogenizer, in that it is satisfactory when the inorganic particles are sufficiently dispersed to provide desired SPF values. When combined with ultramarine pigments, the color will change and may also provide a clear appearance (using the micronized inorganics) as the composition is applied to the skin.

In accordance with a two-vessel process, an aloe or water or aloe/water phase can be prepared by measuring deionized water into a receptacle and mixing. Xanthan gum is sprinkled and mixed until free from lumps. Carrageenan is then mixed in until freed from lumps. (Carrageenan could serve as an alternative to xanthan gum throughout the present disclosure.) The mixture is optionally slowly heated to approximately 80° C., although room temperature or below is preferred. Vegetable glycerin is then added, followed by aloe vera gel. The composite is mixed until completely uniform.

The oil phase is prepared separately in another vessel, at approximately 75° C. Sun-block agents comprising the following are mixed together (with an homogenizer or high speed mixer) until dissolved: refined sunflower oil, lanolin, phospholipids or liposomes, coconut oil, stearic acid, beta carotene, orange wax, beeswax, essential oils, and Vitamin E oil. When mixed, sucrose stearate is slowly added. While maintaining a temperature of 75° C., micronized zinc oxide is sprinkled very slowly and homogenized until smooth and uniform. Cosmetic microspheres and/or titanium dioxide can then be added in the same fashion as the micronized zinc oxide. The temperature of the mixture is raised to 80° C., and the water phase as described above is then added to the oil phase under heavy mixing conditions. Mixing should continue for at least 30 minutes until the mixture is smooth and homogenous. It is preferable to perform the mixing at room temperature or below.

The combined mixture can then be cooled to 45° C. or below. The following ingredients can then be individually added, mixing each well before adding another: aloe vera gel, granular borax, grapefruit seed extract, ascorbyl palmitate, butter milk powder, milk powder. Preservatives are then added and can comprise the following: Biovert® substrate, glucose, lactoperoxidase, and glucose oxidase. Essential oils, for example rosemary oil, peru balsam oil, and olibanum oil (frankincense) are then added to provide fragrance and mixed preferably with homogenization until smooth and homogenous.

An alternative method for formulating the composition is as follows: the formulation is prepared using a two-vessel method, in which the oil and aloe or water or aloe/water phases are individually prepared. In accordance with this two-vessel process, an aloe or water or aloe/water phase is prepared by measuring deionized water and/or aloe into a receptacle and mixing with an homogenizer as it the preferred method through this disclosure. Carrageenan is then mixed in until freed from lumps. The mixture is optionally slowly heated to approximately 80° C., although room temperature or below is preferred. Vegetable glycerin is then added, followed by aloe vera gel. The composite is mixed via homogenizer until completely uniform.

The oil phase is prepared separately in another vessel, at approximately 75° C. Sun-block agents comprising the following are mixed together until dissolved (preferentially ith an homogenizer): jojoba oil, rice bran oil, lanolin, phospholipids or liposomes, stearic acid, orange wax, beeswax, essential oils, and Vitamin E oil. While maintaining a temperature of 75° C., micronized zinc oxide and titanium dioxide are sprinkled very slowly and homogenized until smooth and uniform. The temperature of the mixture is raised to 80° C., and the water phase as described above is then added to the oil phase under heavy mixing conditions. Homogenization mixing should continue for at least 30 minutes until the mixture is smooth and homogenous.

Based on the recent decreases in the North American bee population, beeswax may become impossible to obtain or too expensive to purchase. In today's market, there is a range of waxes based on plants (vegetables or fruits) that are non-paraffinic. It may be necessary to substitute for the beeswax with a plant wax such as;

bayberry wax, candelilla wax, carnauba wax, castor wax, esparto wax, Japan wax (from the berries of Rhus and Toxicodendron), ouricury wax, or rice bran wax.

The combined mixture can then be cooled to 45° C. Again, it is preferable to conduct the mixing at room temperature or below. The following ingredients can then be individually added, mixing each well before adding another: aloe vera gel, grapefruit seed extract, ascorbyl palmitate. Preservatives are then added and can comprise the following: Biovert Substrate®, glucose, lactoperoxidase, and glucose oxidase. Essential oils, for example rosemary oil, peru balsam oil, and olibanum oil (frankincense) are then added to provide fragrance and mixed until smooth and homogenous.

The following examples serve as illustrations of the compositions of the present disclosure, however, they do not limit the scope of the disclosure described herein. Also, the examples below indicate the exact amounts of each constituent to facilitate the preparation of a 1000 gm of the composition. For each constituent, a range of weights and therefore weight percentages can be added to provide a corresponding formulation with similar properties including the ability to change viscosity and consistency of the final composition. Ranges are presented in the corresponding claims.

### EXAMPLE I

211.79 ml of deionized water was added to the receptacle. 0.25 grams of xanthan gum was then added to the receptacle. The composition was mixed until free from lumps. 1.0 g of Carrageenan was added to the receptacle. The composition was mixed until free from lumps. The mixture was heated to 80° C. 15.0 grams of vegetable glycerin was then added to the receptacle, along with 15.0 g of Aloe Vera Gel. The ingredients in the receptacle were then mixed until completely uniform. In a second receptacle, 15.0 g of sunflower oil, 30.0 g of phosphatidyl choline, 1.0 g of coconut oil, 80.0 g of carrier oils, 10.0 g of stearic acid, 0.0005 g of beta carotene, 1.0 g of orange wax, 5.0 g of beeswax, and 0.5 g of vitamin E oil (tocopherol) were mixed until all solids were dissolved, and the mixture was heated to 75° C. 5.0 g Crodesta F-160® (produced by Croda USA) was slowly added, while maintaining the temperature at 75° C. 80.0 grams of micronized zinc oxide (Z-cote®) was sprinkled in slowly and homogenized until smooth and uniform. 10.0 grams of Sensient Cosmetic Microspheres CM-1111 was sprinkled in slowly and homogenized until smooth and uniform. The temperature of this receptacle was increased to 80° C. The first receptacle was then added to the second with vigorous mixing at 80° C. Mixing (preferably by an homogenizer) continued for 30 minutes until the composite was smooth and homogenous. The temperature of the receptacle was lowered to 45° C. 5.0 grams Aloe Vera gel was added, while mixing thoroughly. 0.2 g of borax granular was added, while mixing thoroughly. 5.0 grams of Grapefruit Seed Extract (GSE) was added, while mixing thoroughly. 0.5 grams of ascorbyl palmitate was added, while mixing thoroughly. 0.5 grams of milk powder and 0.5 grams of buttermilk powder were added, while mixing thoroughly. 5.25 grams of Biovert® substrate, a product of Arch Chemicals and a composite of glucose, lactoperoxidase, and glucose oxidase was then added, mixing thoroughly. 1.0 grams of rosemary oil, 0.5 g of peru balsam oil, and 1 gram of olibanum oil (frankincense) were then added. The receptacle was mixed until smooth and homogenous.

SPF (sun protection factor) can be measured as the ratio of the optical signal through the substrate without sunscreen divided by the optical signal through the substrate coated with the sunscreen. The system is calibrated against a series of sunscreens of known SPF (4 through 36) determined in-vivo using the FDA monograph method (Federal Register, Aug. 25, 1978, Sunscreen drug products for over-the-counter human drugs. pp 38206-38269.)

The resulting SPF of the composition of Example I above when measured in-vitro was 31.5 and the composition was aesthetically satisfactory and stable.

### EXAMPLE II

139 ml of deionized water was added to the receptacle. 0.91 g of Carrageenan was added to the receptacle. The composition was mixed until free from lumps. The mixture was heated to 80° C. 13.59 grams of vegetable glycerin was then added to the receptacle. The ingredients in the receptacle were then mixed until completely uniform. In a second receptacle, 22.6 g of rice bran oil, 27.18 g of phosphatidyl choline, 36.24 g of carrier oils, 9.06 g of stearic acid, 0.46 g of orange wax, 11.3 g of beeswax, and 0.91 g of vitamin e oil (tocopherol) were mixed until all solids were dissolved, and the mixture was heated to 75° C. 54.41 grams of micronized zinc oxide (Z-cote®) was sprinkled in slowly and homogenized until smooth and uniform. 21.85 grams of micronized titanium dioxide was sprinkled in slowly and homogenized until smooth and uniform. The temperature of this receptacle was increased to 80° C. The first receptacle was then added to the second with vigorous mixing at 80° C. Mixing continued for 30 minutes until the composite was smooth and homogenous. The temperature of the receptacle was lowered to 45° C. 1.13 grams of Grapefruit Seed Extract (GSE) was added, while mixing thoroughly. 0.453 grams of ascorbyl palmitate was added, while mixing thoroughly. 0.226 grams of Biovert® substrate, a product of Arch Chemicals and a composite of glucose, lactoperoxidase, and glucose oxidase was then added, mixing thoroughly. 0.5 grams of orange oil was added. The receptacle was mixed until smooth and homogenous.

The resulting SPF of the composition of Example II above when measured in vitro was 30.7 and the composition was aesthetically satisfactory and stable.

### EXAMPLE III

139 ml of deionized water was added to the receptacle. 0.91 g of Carrageenan was added to the receptacle. The composition was mixed until free from lumps. The mixture was heated to 80° C. 13.59 grams of vegetable glycerin and 68.0 grams of Aloe Vera gel were then added to the receptacle. The ingredients in the receptacle were then mixed until completely uniform. In a second receptacle, 22.6 g of rice bran oil, 27.18 g of phosphatidyl choline, 36.24 g of carrier oils, 9.06 g of stearic acid, 0.46 g of orange wax, 11.3 g of beeswax, and 0.91 g of vitamin E oil (tocopherol) were mixed until all solids were dissolved, and the mixture was heated to 75° C. 54.41 grams of micronized zinc oxide (Z-Cote®) was sprinkled in slowly and homogenized until smooth and uniform. 21.85 grams of micronized titanium dioxide was sprinkled in slowly and homogenized until smooth and uniform. The temperature of this receptacle was increased to 80° C. The first receptacle was then added to the second with vigorous mixing at 80° C. Mixing continued for 30 minutes until the composite was smooth and homogenous. The temperature of the receptacle was lowered to 45° C. 22.67 grams of Aloe Vera gel was added, while mixing thoroughly. 1.13 grams of Grapefruit Seed Extract (GSE) was added, while mixing thoroughly. 0.453 grams of ascorbyl palmitate was added, while mixing thoroughly. 0.226 grams of Biovert ® substrate, a product of Arch Chemicals and a composite of glucose, lactoperoxidase, and glucose oxidase was then added, mixing thoroughly. 0.5 grams of orange oil was added. The receptacle was mixed until smooth and homogenous.

The resulting SPF of the composition of Example III was measured in vitro to be 30.9 and the composition was aesthetically satisfactory and stable.

### EXAMPLE IV

This example was made in accordance with the method of Example III above, with jojoba oil replacing the rice bran oil in equal quantity by weight. The resulting SPF of the composition of Example IV was tested in vitro to be 30.7 and the composition was aesthetically satisfactory and stable.

### EXAMPLE V

This example was made in accordance with the method of Example IV above, without the addition of micronized titanium dioxide in the composition. The resulting SPF of the composition of Example V was tested in vitro to be 19.6 and the composition was aesthetically satisfactory and stable.

### EXAMPLE VI

This example was made in accordance with the method of Example I above, with twice as much aloe vera gel (a total of 12 g) used in each instance of its addition into the composition. The resulting SPF of the composition of Example VI is in the range of 31.5-33 when measured in vitro and the composition is aesthetically satisfactory.

### EXAMPLE VII

This example was made in accordance with the method of Example I above, with three times as much aloe vera gel (a total of 18 g) used in each instance of its addition into the composition. The resulting SPF of the composition of Example VII is in the range of 31.5-35 when measured in vitro and the composition is aesthetically satisfactory.

### EXAMPLE VIII

This example was made in accordance with the method of Example I above, with four times as much aloe vera gel (a total of 24 g) used in each instance of its addition into the composition. The resulting SPF of the composition of Example VIII is in the range of 31.5-37 when measured in vitro and the composition is aesthetically satisfactory.

### EXAMPLE IX

26.30 ml of deionized water was added to a receptacle. 20.0 grams of Cold Pressed Aloe, 1.75 grams of vegetable glycerin, and 0.25 grams of grapefruit seed extract were mixed into the water. 0.35 g of Xanthan gum was added to the receptacle, with good mixing, until all ingredients were dissolved. The mixture was heated to 40° C. In a second receptacle, 19.2 g of rice bran oil mixed together with 3.5 g of dispersed phosphatidyl choline, 7.0 g of suitable carrier such as castor oil, avocado oil, broccoli seed oil, keratin, micronized or colloidal bentonite, etc. (essential oils or equivalent SPF boosting agents can be used), 0.1 g of orange wax, and 2.5 g of beeswax were mixed until all solids were dissolved, and the mixture was heated to 65° C. When the solution of the second receptacle was heated and became homogenous, 12.0 grams of micronized zinc oxide (Z-Cote®), 4.8 grams of natural source tocopherol (D-alpha), and 4.8 grams of T-Cote® are added to this second receptacle requiring good agitation and maintaining temperature until the micronized powders were properly wetted. A high-energy mixer was used to disperse the ingredients. The first receptacle (water phase) was then added to the second receptacle (oil phase) with high-speed mixing. On a small scale (less than 200 grams), the addition of phases can be reversed. Mixing continued until the composite was cooled. To this mixture, 1.0 gram of Biovert® substrate (a product of Arch Chemicals and a composite of glucose, lactoperoxidase, and glucose oxidase) was then added, mixing thoroughly. 0.05 grams of Biovert® enzyme was added. The receptacle was mixed until smooth and homogenous.

The resulting SPF of the composition of Example IX above when measured in vitro was 30.9 and the composition was aesthetically satisfactory and stable.

### EXAMPLES X-XVIII

Beeswax was heated until melted. Other suitable waxes as described above may be used as substitutes (either individually or in combination). The following ingredients to provide a mixture were then added, in decreasing order of weight: coconut oil, sunflower oil, tocopherol acetate, tocopherol, lanolin, peppermint oil, comfrey root extract, and rosemary extract. No component was added in greater quantity than the initial beeswax. The composition was stirred for several minutes while a constant temperature was maintained above the melting point for beeswax (146 F/62 C). This mixture was then mixed thoroughly preferentially with an homogenizer with each of the above compositions described in Examples I-IX, at a ratio of 90:10 of the current composition to this mixture and then poured into a receptacle and cooled.

| | Current Composition mixed |
|---|---|
| Example # | at 90:10 ratio with Mixture: |
| X | Example 1 |
| XI | Example 2 |
| XII | Example 3 |
| XIII | Example 4 |
| XIV | Example 5 |
| XV | Example 6 |
| XVI | Example 7 |
| XVII | Example 8 |
| XVIII | Example 9 |

### EXAMPLES XIX-XXVII

Beeswax was heated until melted. The following ingredients were then added, in decreasing order of weight: coconut oil, sweet almond oil, tocopheryl acetate, tocopheryl, lanolin, peppermint oil, comfrey root extract, and rosemary extract. Coconut oil was added in greater quantity than the beeswax, all other ingredients in lower quantity. The composition was stirred for several minutes while a constant temperature was maintained above the melting point for beeswax (146 F/62 C). This composition was then mixed thoroughly with the above compositions described in Examples I-IX, at a 90-10 ratio, and then poured into a receptacle and cooled.

| | Current Composition mixed |
|---|---|
| Example # | at a 90-10 ratio with |
| XIX | Example 1 |
| XX | Example 2 |
| XXI | Example 3 |
| XXII | Example 4 |
| XXIII | Example 5 |
| XXIV | Example 6 |
| XXV | Example 7 |
| XXVI | Example 8 |
| XXVII | Example 9 |

### EXAMPLES XXVIII-XXXVI

An oil phase was prepared by combining sunflower oil, stearic acid, coconut oil, beeswax, tocopheryl acetate, orange wax, and beta carotene and stirring the resulting mixture under heat at 80 C until homogeneous. To the oil phase was added sucrose stearate and the resulting mixture was heated, at about 50 C. In a separate container a water phase was prepared by dissolving vegetable glycerin and xanthan gum into deionized water. The water and oil phases were combined. Sodium Borate, Biovert® substrate, and aloe vera gel were added and stirred until homogenous. To the resulting mixture was added fragrance at room temperature and the mixture was allowed to equilibrate overnight. This mixture was then mixed thoroughly preferentially with an homogenizer with each of the above compositions described in Examples IX-XXXVI, at a ratio of 90:10 of the current composition to this mixture and then poured into a receptacle and cooled.

| Example # | Composition + |
|---|---|
| XXVIII | Example 1 |
| XXIX | Example 2 |
| XXX | Example 3 |
| XXXI | Example 4 |
| XXXII | Example 5 |
| XXXIII | Example 6 |
| XXXIV | Example 7 |
| XXXV | Example 8 |
| XXXVI | Example 9 |

### EXAMPLE XXXVII

423.58 ml of deionized water was added to the receptacle. Alternatively, the use of ethyl alcohol derived from plant matter, is allowed. The water is heated to 80° C., the alcohol to a slightly lower temperature to avoid boiling or rapid evaporation. 15.0 grams of vegetable glycerin was then added to the receptacle, along with 30.0 g of Aloe Vera Gel. The receptacle was then mixed until completely uniform—again an homogenizer is desirable. In a second receptacle, 15.0 g of sunflower oil, 30.0 g phosphatidyl choline, 1.0 g of coconut oil, 80.0 g of carrier oils, 10.0 g of stearic acid, 0.0005 g of beta carotene (30% fs), 1.0 g of orange wax, 1.0 to 5.0 g of beeswax, and 0.5 g of vitamin e oil (tocopherol) were mixed by homogenization if possible, until all solids were dissolved, and the mixture was heated to 75° C. Optionally 5.0 g Crodesta F-160 ® (produced by Croda USA) was slowly added, while maintaining the temperature at 75° C. 80.0 grams of micronized zinc oxide (Z-cote®) was sprinkled in slowly and homogenized until smooth and uniform. 10.0 grams of Sensient Cosmetic Microspheres CM-111® was sprinkled in slowly and homogenized until smooth and uniform. The temperature of this receptacle was increased to 80° C. if water and a lower temperature if ethanol is used. The first receptacle was then added to the second with vigorous mixing at 60° C.-80° C. Mixing continued for 30 minutes until the composite was smooth and homogenous. The temperature of the receptacle was lowered to 45° C. 10.0 grams Aloe Vera gel was added, while mixing thoroughly. 0.2 g of borax granular was added, while mixing thoroughly. 5.0 grams of Grapefruit Seed Extract (GSE) was added, while mixing thoroughly. 0.5 grams of ascorbyl palmitate was added, while mixing thoroughly. 0.5 grams of milk powder and 0.5 grams of buttermilk powder were added, while mixing thoroughly. 5.25 grams of Biovert® substrate, a product of Arch Chemicals and a composite of glucose, lactoperoxidase, and glucose oxidase was then added, mixing thoroughly. 1.0 grams of rosemary oil, 0.5 g of peru balsam oil, and 1 gram of olibanum oil (frankincense) were then added. The receptacle was mixed until smooth and homogenous. The composition was cooled and poured into a container allowing for a spray application product.

The foregoing claims serve to describe formulations that complete compositions of this disclosure, however, they do not intend to limit the scope of the disclosure:

1. An SPF composition comprising;
(f) water purified using ozonation, ionization, or distillation or any combination thereof wherein alcohol may be substituted for, or combined with water;
(g) at least one emollient including but not limited to chitosan, and aloe vera gel, individually or in any combination;
(h) an oil component with spf boosting agents including but not limited to; silicone oil and essential oils, butter milk, waxes impregnated with inorganic sun-block or sunscreen agent and organic/inorganic micronized particles, wood powder and bentonite clay, keratin, either individually or in any combination;
(i) at least one inorganic sun-block or sunscreen agent including any metal oxide, glass microsphere, silica and silica compound, and optionally metal oxide pigments with particles that are micronized, submicronized, nanoparticle sized, or otherwise individually or in any combination that can be homogenized in either a water phase, a water-aloe phase, or an oil phase or any phase of said composition;
(j) at least one emulsifier wherein said emulsifier includes but is not limited to a phospholipid and/or liposome or an aloe vera gel or an ester of coconut oil individually or in any combination, for emulsifying said water, water-aloe, or said oil phase;
and wherein any of components (a)-(e) are preferably mixed with an homogenizer and wherein an appropriate thickening agent including but not limited to xanthan gum, carageenan, either individually or in any combination is added as required.

2. The composition of claim 1, wherein optionally at least one ultramarine pigment that imparts a color other than white is added to said composition, and wherein said composition includes a titanium dioxide or zinc oxide or fumed or fused silica or silicon dioxide or micronized glass cosmetic spheres so that when said composition is rubbed into the skin, the color of said composition substantially disappears;
(a) at least one sunscreen active agent in an amount effective to protect skin against the actinic radiation of the sun, preferably including ZnO or Z-Cote®; and any other combination of metal oxides including those with boron,
(b) a cytoprotective substance such as a glucose-rich mannose-containing oligosaccharide obtained from and used with aloe barbadensis Miller as the at least one emollient; and
(c) sufficient water or alcohol to form other than a white colored emulsion; and sufficient dispersion to assure SPF of at least 15 and an SPF booster that shows no appreciable toxicity;
(d) texturing agents providing a powdery and/or dry not oil feel to said composition including but not limited to sodium borate, sucrose stearate, tapioca, powdered milk, silicone oils, and;
(e) optionally carotenoids or other effective antioxidants which are translucent or transparent with respect to said composition.

3. The composition of claim 1 wherein said composition is capable of providing formulas or emulsions that are pastes, lotions, sprays, lip balm, crèmes, and other cosmetic or make-up products.

4. A method for creating the composition of claim 1, comprising:
a) preparing a water phase with a base of water, preferentially deionized water;
b) adding thickeners into said water phase and mixing with an homogenizer until free from lumps;
c) adding carrageenan into the mixture and mixing until free of lumps;
d) heating the mixture to up to 80° C.;
e) adding vegetable glycerin and optionally aloe vera gel and mixing until completely uniform;
f) preparing an oil phase mixture by heating one or more carrier oils to 50° C. or greater;
g) adding a dispersant, SPF boosting oils and/or waxes, and optionally stearic acid, an anti-oxidant, and vitamin E oil, and mixing until all the solids are dissolved;
h) slowly adding sucrose stearate, maintaining temperature;
i) adding inorganic sun-block agents while homogenizing until smooth and uniform;
j) increasing the temperature to up to 80° C. and adding said water phase mixture;
k) mixing until smooth and homogenous;
l) cooling the mixture down to 45° C. or less;
m) adding aloe vera gel, and optionally a salt including sodium borate, grapefruit seed extract, and ascorbyl palmitate, powdered butter milk, and powdered milk;
n) adding preservatives;
o) adding essential oils;
p) mixing until smooth and homogenous.

5. The method of claim 4, wherein said water phase can also be an alcohol phase with an appropriate amount of alcohol such that said composition can be dispensed as a spray.

6. The method of claim 4, wherein said dispersant comprises lanolin, phospholipids, lecithin (phosphatidyl choline), liposomes, or mixtures thereof.

7. The method of claim 4, wherein said thickeners comprise xanthan gum, carrageenan, or combinations thereof.

8. The method of claim 4, wherein said SPF boosting oils and/or waxes comprise coconut oil, orange wax, beeswax, jojoba oil, rice bran oil, sesame oil, safflower oil, almond oil, sweet almond oil, eucalyptus oil, sunflower oil, grape seed oil, hazelnut oil, macadamia oil, rose oil, mandarin oil, chamomile oil, ginger oil, lemon oil, sandalwood oil, basil oil, peppermint oil, hyssop oil, castor oil, broccoli seed oil, avocado oil, keratin, bentonite, pomegranate seed oil, individually or any mixtures thereof.

9. The method of claim 4, wherein said carrier oil comprises coconut oil, jojoba oil, rice bran oil, sesame oil, safflower oil, almond oil, eucalyptus oil, sunflower oil, sweet almond oil, grape seed oil, avocado oil, olive oil, evening primrose oil, walnut oil, peanut oil, macadamia oil, basil oil, peppermint oil, hyssop oil, castor oil, broccoli seed oil, avocado oil, pomegranate seed oil, individually or any mixtures thereof.

10. The method of claim 4, wherein said anti-oxidant comprises beta carotene, beta Glucan, green tea extract, grapefruit seed extract, grape seed extract, grape seed oil, rosehip, individually or any mixtures thereof.

11. The method of claim 4, wherein said sucrose stearate is Crodesta® F-160, said sun-block agents comprise zinc oxide, micronized zinc oxide, including Z-Cote®, titanium dioxide, micronized titanium dioxide, including T-Cote®, cosmetic microspheres including CM-111 AS, micronized oxides including iron oxide, silicon dioxide, magnesium oxide, manganese oxide, silica, silicon dioxide, alumina, aluminum oxides or any single or multiple combination thereof and wherein other possible inorganics include the use of fused or fumed silica or even silicon dioxide, and/or cosmetic microspheres with a primary particle size of less than about 300 nm in diameter and wherein larger particle sizes may also be used if necessary along with special pigments to ensure transparency or at least skin color matching.

12. The method of claim 4, wherein said beeswax or appropriate natural substitute for said beeswax has been impregnated by a sun-block agent selected from the group comprising: zinc oxide, micronized zinc oxide (Z-cote®), titanium dioxide, micronized titanium dioxide (T-cote®), iron oxide, silicon dioxide, magnesium oxide, manganese oxide, silica, alumina, aluminum oxides, cosmetic microspheres (CM-111 AS), or any combination thereof.

13. The method of claim 4, wherein said preservatives comprise Biovert® enzyme, Biovert® substrate, glucose, lactoperoxidase, glucose oxidase, tea tree oil, thyme oil, grapefruit seed extract, tocopherol acetate, or mixtures thereof.

14. The method of claim 4, wherein said essential oils comprise oils of rosemary, peru balsam, olibanum, orange, sunflower, safflower, almond, sesame, rice bran, ylang ylang, eucalyptus, jojoba, coconut, allspice, anise, basil, bay leaf, bergamot, camphor, cedar, celery, chamomile, cassia, citronella, cinnamon, clary sage, clove, cumin, geranium, ginger, grapefruit, hyssop, jasmine, juniper, lavender, lemon, lemon grass, manuka, marjoram, melaleuca, myrrh, nutmeg oil, oregano, patchouli, peppermint, pine, rose, rosewood, common sage, sandalwood, spearmint, tangerine, tea tree, thyme, wintergreen individually or any mixtures thereof and wherein said essential oils may be used to provide any fragrance for said composition and wherein sunflower oil may be replaced with sweet almond oil or rice bran oil.

15. The sun-block agents of claim 4, wherein said agents are used in a proportion between 0% and 25% by weight, preferably between 4% and 20% by weight, and most preferably between 4.5% and 18% by weight and wherein said dispersant is used in a proportion between 0% and 20%, preferably between 2% and 10%, and more preferably between 4% and 8%.

16. The method of claim 4, wherein said aloe vera gel comprises aloe barbadensis Miller and more preferably a single species of aloe barbadensis Miller and most preferably aloe barbadensis Miller-Stockton.

17. The method of claim 4, wherein an inorganic sun-block agent is added to said aloe vera gel or an aloe vera liquid, said gel or liquid preferentially cold pressed, prior to addition to said composition.

18. The composition of claim 1, wherein said composition has a pH of at least 5.

19. The composition of claim 1, having a Sun Protection Factor (SPF) of at least 15, and preferably an immuno-responsiveness factor (IRF) of greater than zero and wherein said composition is preferably not toxic in that said composition will not kill cells.

20. The composition of claim 1, wherein said composition is homogenized with a mixture comprising beeswax, coconut oil, sunflower oil, tocopheryl acetate & tocopheryl, lanolin, peppermint oil, comfrey root extract, rosemary extract, or mixtures thereof, and wherein said composition is up to 99 percent by weight of the total new composition comprising said composition and said mixture.

21. The composition of claim 1, wherein said composition is homogenized with a mixture comprising water, sunflower oil, stearic acid, vegetable glycerin, coconut oil, beeswax, tocopheryl acetate (vitamin E), orange wax, aloe barbadensis, rosemary extract, sodium borate (natural borax), xanthan gum (natural thickener), sucrose stearate (sugar emulsifier), glucose and glucose oxidase and lactoperoxidase (sugar and natural enzymes), beta-carotene, green tea extract, fragrance, or mixtures thereof and wherein said composition is up to 99 percent by weight of the total new composition comprising said composition and said mixture.

22. The composition of claim 1, wherein the amount of water, and/or alcohol, and/or aloe vera, are increased by a factor of 2 to 4 by weight.

23. The composition of claim 1, wherein the amount of water, and/or alcohol, and/or aloe vera, are increased until the viscosity of the composition is low enough to be sprayed through a nozzle and wherein said composition will does not clog said nozzle.

24. A composition comprising;
a water phase mixture including a base of water, preferably deionized water;
cold pressed aloe vera gel or liquid, vegetable glycerin, and grapefruit seed extract;
and a thickener or thickening agent; preferably mixed together using an homogenizer for mixing an oil phase mixture that is comprised of essential oils, carrier oils, dispersants, emulsifiers such as phospholipids, SPF boosting oils and/or waxes, and beeswax or a suitable natural alternative, micronized metal or metal oxide powders, including but not limited to metals of zinc, titanium, magnesium, manganese, aluminum, boron, silicon, as well as any or all oxides or combinations of said metals of either micron sized or submicron sized particles, optionally anti-oxidizing, free-radical scavenging agents including carotenoids and green tea extract, preservatives including peroxidase enzymes.

25. The composition of claim 24, wherein said sucrose stearate is Crodesta® F-160, said sun-block agents comprise zinc oxide, micronized zinc oxide, including Z-Cote®, titanium dioxide, micronized titanium dioxide, including T-Cote®, cosmetic microspheres including CM-111 AS, micronized oxides including iron oxide, silicon dioxide, magnesium oxide, manganese oxide, silica, silicon dioxide, alumina, aluminum oxides or any single or multiple combination thereof and wherein other possible inorganics include the use of fused or fumed silica or even silicon dioxide, and/or cosmetic microspheres with a primary particle size of less than about 300 nm in diameter and wherein larger particle sizes may also be used if necessary along with special pigments to ensure transparency or at least skin color matching.

26. A method for creating the composition of claim 24, comprising;
a) preparing a water phase with a base of deionized water;
b) adding Aloe, vegetable glycerin, and grapefruit seed extract;
c) adding a thickener with good mixing until all ingredients are dissolved;
d) heating the mixture to 40° C.;
e) preparing an oil phase mixture by combining carrier oils, dispersants, emulsifiers, SPF boosting oils and/or waxes, and beeswax until all solids were dissolved, and heating to 65° C.;
f) adding SPF agents and tocopherol with good agitation while maintaining temperature, until the micronized powders were properly wetted;
g) adding the water phase mixture to the oil phase mixture with high-speed mixing until the composite was cooled;
h) adding preservatives;
i) mixing until smooth and homogenous.

27. The method of claim 26, wherein said water phase can also be an alcohol phase with an appropriate amount of alcohol such that said composition can be dispensed as a spray and wherein said dispersants comprise lanolin, phospholipids, lecithin (phosphatidyl choline), liposomes, or mixtures thereof and wherein said thickener comprises xanthan gum, carrageenan, individually or any combination thereof.

28. The method of claim 26, wherein said SPF boosting oils and/or waxes comprise coconut oil, orange wax, beeswax, jojoba oil, rice bran oil, sesame oil, safflower oil, almond oil, sweet almond oil, eucalyptus oil, sunflower oil, grape seed oil, hazelnut oil, macadamia oil, rose oil, mandarin oil, chamomile oil, ginger oil, lemon oil, sandalwood oil, basil oil, peppermint oil, hyssop oil, castor oil, broccoli seed oil, avocado oil, keratin, bentonite, pomegranate seed oil, individually or any mixtures thereof and wherein said carrier oil comprises coconut oil, jojoba oil, rice bran oil, sesame oil, safflower oil, almond oil, eucalyptus oil, sunflower oil, sweet almond oil, grape seed oil, avocado oil, olive oil, evening primrose oil, walnut oil, peanut oil, macadamia oil, basil oil, peppermint oil, hyssop oil, castor oil, broccoli seed oil, avocado oil, pomegranate seed oil, individually or any mixtures thereof and wherein said anti-oxidant comprises beta carotene, beta Glucan, green tea extract, grapefruit seed extract, grape seed extract, grape seed oil, rosehip, individually or any mixtures thereof.

29. The method of claim 26, wherein said preservatives comprise Biovert® enzyme, Biovert® substrate, glucose, lactoperoxidase, glucose oxidase, tea tree oil, thyme oil, grapefruit seed extract, tocopherol acetate, green tea extract, individually, or mixtures thereof.

30. The composition of claim 24, wherein said essential oils comprise oils of rosemary, peru balsam, olibanum, orange, sunflower, safflower, almond, sesame, rice bran, ylang ylang, eucalyptus, jojoba, coconut, allspice, anise, basil, bay leaf, bergamot, camphor, cedar, celery, chamomile, cassia, citronella, cinnamon, clary sage, clove, cumin, geranium, ginger, grapefruit, hyssop, jasmine, juniper, lavender, lemon, lemon grass, manuka, marjoram, melaleuca, myrrh, nutmeg oil, oregano, patchouli, peppermint, pine, rose, rosewood, common sage, sandalwood, spearmint, tangerine, tea tree, thyme, wintergreen individually or any mixtures thereof and wherein said essential oils may be used to provide any fragrance for said composition.

31. The sun-block agents of claim 24, wherein said agents are used in a proportion between 0% and 25% by weight, preferably between 4% and 20% by weight, and most preferably between 4.5% and 18% by weight and wherein said dispersant is used in a proportion between 0% and 20%, preferably between 2% and 10%, and more preferably between 4% and 8%.

32. The method of claim 26, wherein said aloe vera gel comprises aloe barbadensis Miller and more preferably a single species of aloe barbadensis Miller and most preferably aloe barbadensisMiller-Stockton.

33. The method of claim 26, wherein an inorganic sun-block agent is added to said aloe vera gel or an aloe vera liquid, said gel or liquid preferentially cold pressed, prior to addition to said composition.

34. The composition of claim 24, wherein said composition has a pH of at least 5.

35. The composition of claim 24, having a Sun Protection Factor (SPF) of at least 15, and preferably an immuno-responsiveness factor (IRF) of greater than zero and wherein said composition is preferably not toxic in that said composition will not kill cells.

36. The composition of claim 24, wherein said composition is homogenized with a mixture comprising beeswax, coconut oil, sunflower oil, tocopheryl acetate & tocopheryl, lanolin, peppermint oil, comfrey root extract, rosemary extract, or mixtures thereof, and wherein said composition is up to 99 percent by weight of the total new composition comprising said composition and said mixture.

37. The composition of claim 24, wherein said composition is homogenized with a mixture comprising water, sunflower oil, stearic acid, vegetable glycerin, coconut oil, beeswax, tocopheryl acetate (vitamin E), orange wax, aloe barbadensis, rosemary extract, sodium borate (natural borax), xanthan gum (natural thickener), sucrose stearate (sugar emulsifier), glucose and glucose oxidase and lactoperoxidase (sugar and natural enzymes), beta-carotene, fragrance, or mixtures thereof and wherein said composition is up to 99 percent by weight of the total new composition comprising said composition and said mixture.

38. The composition of claim 24, wherein the amount of water, and/or alcohol, and/or aloe vera, are increased by a factor of 2 to 4 by weight.

39. The composition of claim 24, wherein the amount of water, and/or alcohol, and/or aloe vera, are increased until the viscosity of the composition is low enough to be sprayed through a nozzle and wherein said composition will does not clog said nozzle.

40. The method of claim 26, wherein an inorganic sun-block agent is added to said aloe vera gel or an aloe vera liquid, said gel or liquid preferentially cold pressed, prior to addition to said composition.

41. The composition of claim 24, having a Sun Protection Factor (SPF) of at least 15, and preferably an immuno-responsiveness factor (IRF) of greater than zero and wherein said composition is preferably not toxic in that said composition will not kill cells.

42. The composition of claim 24, wherein a carrier oil which is more preferably an essential oil is present in said composition in an amount of between about 0.1% and about 10%, more preferably, it should be present in the amount of between about 1% and about 5%, most preferably, it should be present in the amount of between about 2% and about 4%, wherein essential oils include oils of jojoba, rice bran, sesame, safflower, almond, sweet almond, eucalyptus, sunflower, peru balsam, rosemary, olibanum, orange, sunflower, ylang ylang, apricot kernel, avocado, borage, cocoa butter, evening primrose, grapeseed, hazelnut, kukui, macademia nut, olive, peanut, pecan, rose hip, bergamot, jasmine, neroli, patchouli, petitgrain, rose, vetiver, chamomile, mandarin, lavender, grapefruit, cypress, bay laurel, frankincense, clary sage, ginger, helichrysum, lemon, sandalwood, basil, black pepper, peppermint, geranium, wintergreen, thyme, tea tree, tangerine, spearmint, common sage, rosewood, pine, patchouli, oregano, nutmeg, myrrh, melaleuca, marjoram, manuka, lemon grass, lavender, juniper, ginger, cumin, clove, camphor, bay leaf, anise, allspice, and hyssop.

43. The composition of claim 24, wherein an emollient present in said composition in a ratio to the carrier concentration of from about 1:1 to about 3:1, most preferably, about 2:1 and wherein said carrier oil and the emollient should comprise from about 2% to about 40% of the total composition weight
and wherein other possible inorganics include the use of fused or fumed silica or even silicon dioxide, and/or cosmetic microspheres with a primary particle size of less than about 300 nm in diameter and wherein larger particle sizes may also be used if necessary along with special pigments to ensure transparency or at least skin color matching.

44. The composition of claim 24, wherein said inorganics are present in the amount of from about 2% to about 25%, more preferably, present in the amount of from about 2% to about 20%, and wherein said composition is oil dispersible, and wherein said inorganic may or may not include a surface coating.

45. The composition of claim 24, wherein the ratio of titanium dioxide or zinc oxide to the weight of the carrier oil and the emollient combined should be from about 0.0:1 to about 1:1, and more preferably, the ratio should be between about 1.0:0.25 to 1.0:0.33 or 3.0:2.0.

46. The composition of claim 1, wherein a polymeric thickener/stabilizer, including hydrolyzed wheat protein as well as additional emollient oils, microbial preservatives, waterproofing agents, antioxidants, fragrance, humectant, and a water vehicle may all be utilized.

47. The composition of claim 1, wherein said composition is a base formulation used as carrier compositions for active topical agents having dermatological effects, including depigmentation agents, anti-aging ingredients, anti-fungal agents, anti-microbial agents, wherein depigmentation agents include magnesium ascorbyl phosphate or hydroquinone, anti-aging agents include retinoid compounds and alpha-hydroxy acids, anti-fungal agents include azole compounds including ketoconazole, anti-microbial agents may include triclosan.

48. The composition of claim 1, wherein aloe can be used as both an emollient and a surfactant/dispersion agent together with either ZnO, titanium dioxide, silicon dioxide, fluoropolymers, silica, magnesium, glass microspheres, clays, wood powders, micronized or otherwise (inorganic or organic sun-block agents) with optional additions of anti-oxidants including caretonoids, resveratrol, bee pollen, flower pollen, and dispersing agents including phosphotidyl choline or phospholipids, or liposomes or keratins.

49. A composition wherein SPF values of 15 or greater are achieved by blending and subsequent mixing of aloe with vegetable glycerin and zinc oxide, said zinc oxide may be micronized, wherein mixing is accomplished in a aloe-water phase that is subsequently mixed at high speed with an oil-phase, and wherein blending or homogenizing is accomplished using either said aloe-water phase or said oil phase and said aloe would not be necessarily diluted with water until after full addition and blending of said inorganic sun-block agents and wherein water dilution during or after blending is acceptable but not necessary and in some cases is undesirable.

50. The composition of claim 1, wherein rosehip, called known as rose haw, the pomaceous fruit of the rose plant and a powerful antioxidant is added and contains Vitamin C, vitamins A, D and E, and antioxidant flavonoids.

51. The composition of claim 1, wherein said composition may comprise one or more additional ingredients, including additional emollients waterproofing agents, dry-feel/powdery-feel modifiers, antimicrobial preservatives and/or fragrances.

52. The composition of claim 1, wherein said compositions may also comprise liposomes, lanolin, and/or lecithin.

53. The composition of claim 1, wherein said compositions may also comprise collagen, elastin, panthenol (pro-vitamin B5) and keratin, including humectants such as glycerine and phospholipids.

54. The composition of claim 1, wherein a carrier oil may be selected from the group comprising essential oils or other known non-toxic esters and includes castor oil, avocado oil, broccoli seed oil, jojoba and avocado oils, keratin, and micronized or colloidal bentonite, and wherein said carrier oil and said emollient comprise from about 1% to about 40% of the total weight of said compositions.

55. The composition of claim 1, wherein said compositions include Tocopherol, or Vitamin E oil, a fat-soluble vitamin that may be present in any of eight forms and optionally include preservatives comprising the following: Biovert® substrate, glucose, lactoperoxidase, and glucose oxidase together with essential oils, including rosemary oil, peru balsam oil, and olibanum oil (frankincense) added to provide fragrance and mixed with homogenization until smooth and homogenous.

56. A composition comprising a water phase mixture including a base of water, preferably deionized, distilled or ozonated water; cold pressed aloe vera gel or liquid, vegetable glycerin, and grapefruit seed extract; and a thickener or thickening agent; preferably mixed together with an homogenizer of an oil phase mixture that is comprised of essential oils, carrier oils, dispersants, emulsifiers such as phospholipids, SPF boosting oils and/or waxes, and beeswax or a suitable natural alternative, micronized metal or metal oxide powders, including magnesium stearate and magnesium oxide, including but not limited to metals of zinc, titanium, magnesium, manganese, aluminum, boron, silicon, as well as any or all oxides or combinations of said metals of either micron sized or submicron sized particles, optionally anti-oxidizing, free-radical scavenging agents such as carotenoids, preservatives including enzymes such as Biovert® substrate, glucose, lactoperoxidase, and glucose oxidase.

57. The compositions of claim 1, comprising any individual component or any combination of components including; selenium, botanical butters, extracts of cucumber, chamomile, eyebright, pycnogenol, marine collagen, lax oil, witch hazel, propolis, omega-3 fatty acids, beta-carotene, beta glucan, vitamin C, arnica, almond oil, primrose oil, astragalus, sasparilla, ligustrum, Echinacea, dandelion leaf, royal jelly or any of the known amino acids.
